(19) United States
(12) Patent Application Publication
(10) Pub. No.: US 2003/0096265 A1
(43) Pub. Date: May 22, 2003

(54) Phosphoramidites for coupling oligonucleotides to [2 + 2] photoreactive groups

(75) Inventors: Charles K. Brush, Whitefish Bay, WI (US); Robert Elghanian, Skokie, IL (US); Yanzheng Xu, Redwood Shore, CA (US)

Correspondence Address: BRINKS HOFER GILSON & LIONE, P.O. Box 10395, Chicago, IL, 60610, US

(73) Assignee: Motorola, Inc.

(21) Appl. No.: 10/185,279

(22) Filed: Jun. 28, 2002

Related U.S. Application Data
(63) Continuation-in-part of application No. 09/928,250, filed on Aug. 9, 2001.
Application No. 09/928,250 is a continuation-in-part of application No. 09/344,620, filed on Jun. 25, 1999, now Pat. No. 6,372,813.

(51) Int. Cl. C12Q 1/68; C07F 9/02; C07F 9/6512

(52) U.S. Cl. 435/6; 544/243; 544/244; 558/199

(57) ABSTRACT

Photoreactive phosphoramidites useful for attaching photoreactive sites to nucleic acids and oligonucleotides are synthesized. The resultant nucleic acid or oligonucleotide probes incorporating the photoreactive sites are then attached to a polymer-coated support by a [2+2] cycloaddition to form a microarray.

### REFERENCE TO RELATED APPLICATIONS

This application is a continuation-in-part of U.S. Nonprovisional Application No. 09/928,250, filed Aug. 9, 2001, entitled “The Use and Evaluation of 2+2 Photoaddition in Immobilization of Oligonucleotides on A Three Dimensional Hydrogel Matrix,” (incorporated by reference) which is a continuation-in-part of U.S. Nonprovisional Application No. 09/344,620, filed Jun. 25, 1999, entitled “Methods and Compositions for Attachment of Biomolecules to Solid Supports, Hydrogels, and Hydrogel Arrays” (incorporated by reference).

### BACKGROUND

Chip based DNA microarrays are an integration of circuit fabrication technology and genetics. DNA microarrays consist of matrices of DNA arranged on a solid surface where the DNA at each position recognizes the expression of a different target sequence. Microarrays are used to identify which genes are turned on or off in a cell or tissue, and to evaluate the activity level under various conditions. This knowledge enables researchers to determine whether a cell is diseased or the effect of a drug on a cell or group of cells. Such studies are critical to determine a drug's efficacy or toxicity, to identify new drug targets, and to more accurately diagnose illnesses, such as specific types of cancer. Additionally, the technology is useful to classify tumors with the hope of establishing a correlation between a specific type of cancer, the therapeutic regiment used for treatment, and survival.

Photolithography technology, similar to that employed for transistor etching into silicon chips, is often used to layer chains of nucleotides, the basic units of DNA, onto silicon. Additionally, oligonucleotides, often referred to as “probes,” may be deposited onto solid substrates, or solid substrates coated with various polymers. Various deposition or spraying methods are used to deposit the nucleotides, including piezoelectric technology similar to that used for ink-jet printer heads and robotic methods. The probes are attached to the substrates or polymers by thermal, chemical, or light-based methods to form the microarray.

The genes of interest, or “targets,” are generally put into solution in a “fluidics station” which disperses the target solution on the microarray surface. If fluorescence detection is used, the targets may be tagged with fluorescent labels. Nucleotide targets which are complementing, or “recognized” by, the nucleotide containing probes on the support or polymer then bind, or hybridize, with their corresponding probes. Additionally, the targets may be enzymatically tagged after hybridization to their respective probes. After rinsing to remove any unbound targets from the microarray, the presence and or concentration of specific targets may be determined by spectroscopic or other methods.

Many beneficial applications exist for microarrays, including diagnosing mutations in HIV-1, studying the gene defects which lead to cancer, polymorphism screening and genotyping, and isolating the genes which lead to genetic based disorders, such as multiple sclerosis.

A microarray may be formed by coating a solid support with a polymer. Acrylamide (CH₂═CHC(O)NH₂; C.A.S. 79-06-1; also known as acrylamide monomer, acrylic amide, propenamide, and 2-propenamide) is an odorless, free-flowing white crystalline substance that is used as a chemical intermediate in the production and synthesis of polyacrylamide polymers. Polyacrylamides have a variety of uses and can be modified to optimize nonionic, anionic, or cationic properties for specified uses, such as a polymer coating for the solid support of a microarray, and to allow for the inclusion of modified functional groups for the attachment of probes. The probes, such as DNA, are later attached.

Chemical immobilization of biomolecules, such as DNA, RNA, peptides, and proteins, on a solid support or within a matrix material, such as a hydrogel, has become a very important aspect of molecular biology research. This is especially true in the manufacturing and application of microarray or chip-based technologies where biomolecules are immobilized as probes.

For polyacrylamide, the necessary functionality for probe attachment presently entails chemical modification of the hydrogel through the formation of amide, ester, or disulfide bonds after polymerization and crosslinking of the hydrogel. An unresolved problem with this approach is the less than optimal stability of the attachment chemistry over time, especially during subsequent manufacturing steps, and under use conditions where the microarray is exposed to high temperatures, ionic solutions, and multiple wash steps. Such conditions promote continued depletion in the quantity of probe molecules present in the array, thus reducing its performance and useful life. A further problem is the low efficiency of the method.

A more recent method has employed direct co-polymerization of an acrylamide-derivatized oligonucleotide. For instance, ACRYDITE (Mosaic Technologies, Boston, Mass.) is an acrylamide phosphoramidite that contains an ethylene group capable of free radical polymerization with acrylamide. Acrydite-modified oligonucleotides are mixed with acrylamide solutions and polymerized directly into the gel matrix (Rehman et al., Nucleic Acids Research, 27, 649-655 (1999). This method still relies on acrylamide as the monomer. Depending on the choice of chemical functionality, similar problems in the stability of attachment, as with the above-mentioned methods, also result.

The present invention seeks to overcome some of the aforesaid disadvantages of the prior art, including the problems associated with chemical attachment of the probes to the polymer-coated support, for the purpose of forming microarrays.

### BRIEF SUMMARY

Microarrays are constructed by covalently bonding synthetic oligonucleotide probes to hydrogels using [2+2] cycloaddition chemistry. Phosphoramidite functionality is incorporated with photoreactive sites to form photoreactive phosphoramidites. The phosphoramidite functionality of the photoreactive phosphoramidites is used to incorporate the photoreactive sites into oligonucleotides. These photoreactive oligonucleotides, or “probes” are attached by [2+2] cycloaddition to a polymer or hydrogel that also incorporates photoreactive sites. Cycloaddition occurs when a hydrogel/probe combination is exposed to ultraviolet light. This cycloaddition results covalent attachment of the probes to the hydrogel, forming a microarray.

### BRIEF DESCRIPTION OF THE DRAWINGS

FIG. 1 shows some photoreactive phosphoramidites useful for incorporating photoreactive sites into oligonucleotides.

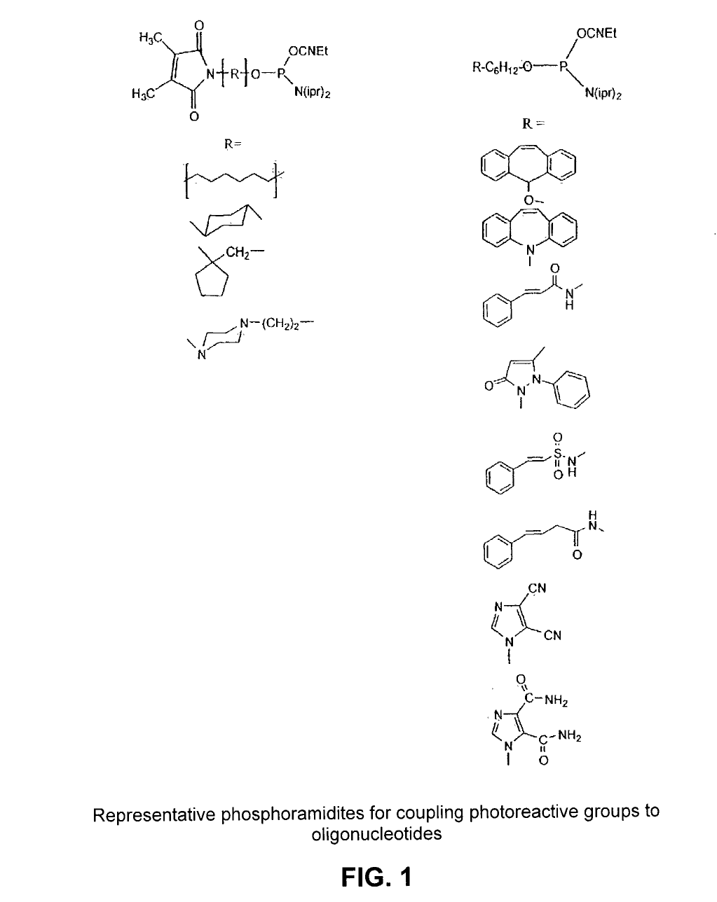

FIG. 2 shows a preferred reaction scheme for incorporating a photoreactive site into an oligonucleotide and attaching the resultant photoreactive probe to an acrylamide hydrogel functionalized with a photoreactive site.

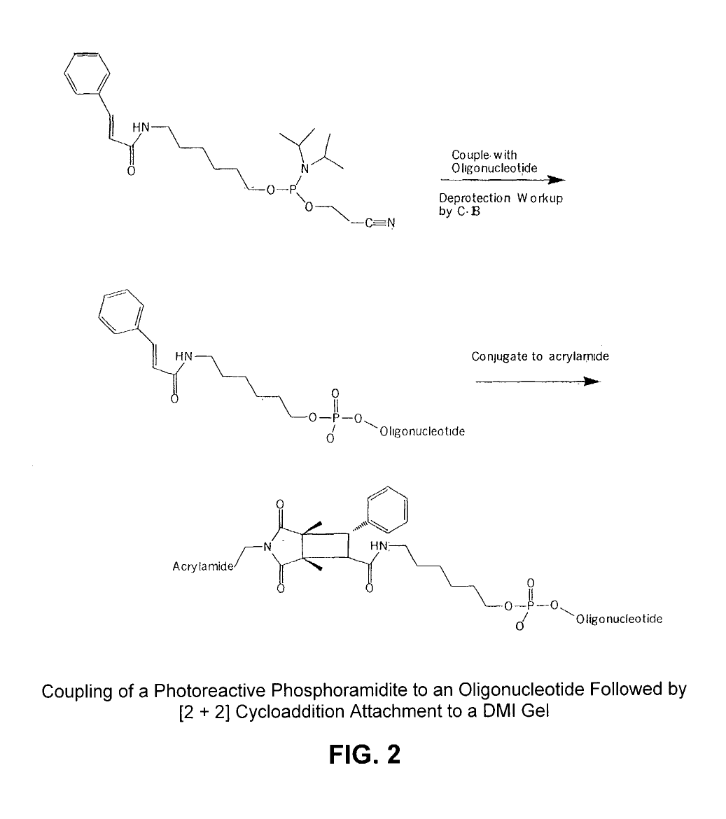

### DETAILED DESCRIPTION

A novel method of incorporating [2+2] photoreactive sites into oligonucleotides using photoreactive phosphoramidites is disclosed. Hydrogel microarrays are formed by polymerizing acrylamide in a controlled fashion to obtain a “prepolymer.” The prepolymer may then be coated on a solid support, such as a glass microscope slide and photochemically crosslinked. Using [2+2] cycloaddition chemistry, photoreactive oligonucleotide probes, including DNA, RNA, and modifications thereof, may be attached.

For the [2+2] cycloaddition to occur, the prepolymer and probes contain photoreactive sites, which are inherent or added by chemical means, which form covalent bonds upon irradiation with light. The oligonucleotides or polynucleotides are functionalized with phosphoramidite couplers that include a first photoreactive site capable of undergoing [2+2] cycloaddition, thus forming photoreactive probes. Additionally, the hydrogel polymer support includes a second photoreactive site that can undergo [2+2] cycloaddition. When irradiated with ultraviolet light at an appropriate wavelength, the probes attach to the hydrogel by [2+2] cycloaddition between the first and second photoreactive sites, respectively.

Generally, microarrays are a collection of probe binding sites at known physical locations on a surface. By positioning tiny specks of probe molecules at known surface locations and then exposing a collection of target molecules to the probes, selective binding occurs between specific probes and targets. For example, because adenine only binds to thymine, a thymine probe will selectively bind to an adenine target.

Once probe/target binding occurs, unbound targets are washed away and the microarray is analyzed to determine which targets have bound at specific probe locations on the microarray. If an internal standard is included with the targets, and probes are provided for the standard to bind with on the microarray, quantitative determinations may also be made. Because many different probes can be deposited on a single microarray, numerous types of binding analyses can be performed simultaneously.

While the invention may be used to form any type of array in which probes are attached to a support by [2+2] cycloaddition chemistry, common arrays include expression, single nucleotide polymorphism (SNP), and protein microarrays. Additionally, the photoreactive probes may be attached to other species, including labels and linkers, capable of undergoing [2+2] photocycloaddition.

Expression/Targets

Expression microarrays are used to detect the presence of nucleic acids or polynucleotides generated, or expressed, by genes. These nucleic acids, or “targets,” are preferably polynucleotides such as RNA (including mRNA). They may be taken from any biological source, including pathogens, healthy or diseased tissue or cells, and tissues or cells that have been exposed to drugs. Because expression microarrays are often used to determine if a tissue is expressing different biomolecules than normal due to disease or drug treatment, the targets of interest are often nucleotides produced by these tissues. When targets include mRNA, probes preferably include polynucleotides. When targets include proteins, probes preferably include protein binding molecules, such as other proteins, antibodies (mono- or polyclonal, or recombinant) or nucleic acids, such as aptamers. Other biomolecules, such as carbohydrates, lipids, and small molecules can be detected by antibodies and aptamers.

Standards

In addition to determining the presence of a specific nucleic acid or protein, microarrays may be used to simultaneously make a quantitative determination of the detected targets. This is possible by incorporating “probe standards” into the microarray which selectively bind to specific “target standards,” but do not interfere with analyte probe/target binding. Preferred target standards are yeast mRNA and bacterial mRNA, or combinations thereof. Yeast mRNA is most preferred.

Labels

In an expression microarray, the targets of interest may be labeled with dyes or other fluorophores that fluoresce when irradiated with light of a known wavelength. The labels are attached to the targets by standard chemical/enzymatic methods known to one of skill in the art, as found in Lockhart, et al., Nature Biotechnology, 14, 1675-80 (1996), for example. The fluorescent emission from the labeled nucleic acids allows their detection by spectroscopic methods. By scanning the expression microarray with light at the excitation wavelength or wavelengths of the dyes used, the labeled nucleic acids may be detected. By placing different dyes on different targets, multiple determinations may be made from a single microarray. If photoreactive sites are present or incorporated into the labels, they may be attached to photoreactive nucleotides by [2+2] cycloaddition.

The literature contains examples of many fluorescent dyes suitable for labeling the targets. Preferred labels include those sold under the tradename ALEXA FLUOR. These fluorophores are dyes with trade secret compositions which may be purchased from Molecular Probes, Inc. (849 Pitchford Avenue, Eugene, Oreg. 97402-9165 USA). Of the ALEXA FLUOR dyes, ALEXA-647 is most preferred.

Other preferred labels include the cyanine dyes prepared with succinimidyl ester reactive groups, such as Cy-3, Cy-5, and Cy-5.5. The number immediately after the “Cy” indicates the number of bridge carbons. The number following the decimal point indicates a unique dye structure, which is determined by the substituents on the structure. Cy-3, Cy-5, and Cy-5.5 are available from Amersham Pharmacia Biotech (Piscataway, N.J. USA). Of the cyanine dyes, Cy-3 is most preferred.

SNP

Generally, single nucleotide polymorphism (SNP) microarrays are similar to expression microarrays, including their use of oligonucleotide probes and nucleic acid targets. However, significant differences can exist regarding how fluorescent labels are attached to the targets and how the microarrays are developed. In one aspect of an expression microarray, the targets are labeled prior to their dispersion on the microarray. In one aspect of an SNP array, in which the targets are not previously labeled, the target solution contains non-labeled targets, an active enzyme, a fluorescently labeled nucleoside triphosphates terminator, and optionally, target standards. In this manner, the fluorescent label may be attached to the probe-target duplex after hybridization through enzymatic extension using a polymerase and a nucleotide.

While expression microarrays rely on selective probe/target binding to generate a fluorescent pattern on the array, some SNP microarray methods rely on enzyme selective single base extension (SBE) of a selected probe/target complex. During development of the SNP microarray, the targets bind to their respective probes to form a complex, generally having a double-helix structure. If an appropriate complex is recognized by the active enzyme, it transfers the label by a SBE reaction from the carrier (ddNTP*) to the complex. Thus, fluorescent probe/target sites are selectively created. The SNP microarray may then be washed and scanned similarly to an expression array to confirm the presence of a specific target, and optional quantitation, if probe and target standards are used.

Solid Support

Generally, the polymer or polyacrylamide reactive prepolymer is coated onto a solid support. Preferably, the “solid support” is any solid support that can serve as a support for the polyacrylamide prepolymer, including film, glass, silica, modified silicon, ceramic, plastic, or polymers such as (poly)tetrafluoroethylene, or (poly)vinylidenedifluoride. More preferably the solid support is a material selected from the group consisting of nylon, polystyrene, glass, latex, polypropylene, and activated cellulose. Most preferably, the solid support is glass.

The solid support can be any shape or size, and can exist as a separate entity or as an integral part of any apparatus, such as beads, cuvettes, plates, and vessels. If required, the support may be treated to provide adherence of polyacrylamide to the glass, such as with γ-methacryl-oxypropyl-trimethoxysilane (“Bind Silane,” Pharmacia). In particular, covalent linkage of polyacrylamide hydrogel to the solid support can be done as described in European Patent Application 0 226 470, incorporated by reference. The solid support may optionally contain electronic circuitry used in the detection of molecules, or microfluidics used in the transport of micromolecules. Additionally, if photoreactive sites are present or incorporated into the solid support, photoreactive nucleotides may be attached to the solid support by [2+2] cycloaddition.

Polymer

Preferably, the solid support is coated with a polymer, including acrylamide prepolymer, which may be coated and imaged using standard commercial equipment. Conversion of the prepolymer into a three-dimensional polyacrylamide hydrogel array, or crosslinking, may entail additional steps, including developing the pattern in the array and removing any uncrosslinked polymer. The prepolymer can be functionalized with a photoreactive site before, during, or after it is formed into a hydrogel. A detailed description of polyacrylamide hydrogels and hydrogel arrays made from polyacrylamide reactive prepolymers is given in WO 00/31148, entitled “Polyacrylamide Hydrogels and Hydrogel Arrays Made from Polyacrylamide Reactive Prepolymers.”

Preferably, the polymer is a polymer or copolymer made of at least two co-monomers that form a three-dimensional hydrogel, wherein at least one of the co-monomers can react by [2+2] cycloaddition. Alternatively, the polymer is a polymer or copolymer that forms a three-dimensional hydrogel which is then chemically modified to contain a photoreactive site that undergoes [2+2] cycloaddition.

Most preferably, the polymer is an acrylamide reactive prepolymer made by polymerizing acrylamide with a compound including dimethyl maleimide (DMI), a six carbon linker, and a polymerizable group, such as acrylate, to give a low molecular weight polymer. While not wishing to be bound by any particular theory, it is thought that when the reactive prepolymer is later crosslinked to form a three-dimensional hydrogel, the polymerizable group attaches to the acrylamide to form the hydrogel and the dimethyl maleimide attaches the resultant hydrogel to the solid support, and optionally to the probe if crosslinking and probe attachment are performed concurrently. During this process, it is believed that about 50% of the photoreactive sites on the DMI remain available for further reaction, such as probe attachment.

Probes

Probes are covalently attached to the polymer to form the microarray by [2+2] cycloaddition between a first photoreactive site on the probe and a second photoreactive site on the polymer or reactive prepolymer. Preferable probes include nucleic acids or fragments thereof containing less than about 5000 nucleotides, especially less than about 1000 nucleotides. Most preferably, a probe includes an oligonucleotide, such as DNA, RNA, PNA, or modifications thereof. Probes may be tissue or pathogen specific. Preferably, probes are nucleotides that include a photoreactive site incorporated through a phosphoramidite coupler.

A detailed description of suitable probes, photoreactive sites, and applicable probe modifications to allow [2+2] cycloadditions is given in U.S. patent application Ser. No. 09/344,620, filed Jun. 25, 1999, entitled “Method and Compositions for Attachment of Biomolecules to Solid Supports, Hydrogels and Hydrogel Arrays,” incorporated by reference.

Generally, probe synthesis entails the stepwise addition of nucleoside phosphoramidites to a synthesis support. Nucleoside phosphoramidites are monomers which include a nucleoside and phosphoramidite functionality. Synthesis supports are any support to which a nucleoside may be attached that allows nucleotide synthesis. Preferable synthesis supports include glass or plastic that has been chemically treated for nucleoside attachment.

After the complete oligonucleotide is synthesized on the support, the phosphoramidite incorporating the photoreactive site is coupled to the oligonucleotide to form a photoreactive oligonucleotide or probe. The probe is then removed from the synthesis support, deprotected, and purified. At this time, the photoreactive site is integrated into the oligonucleotide.

During oligonucleotide synthesis, failures at each step are capped. Therefore, only the full-length material has the photoreactive site. Because probes containing the photoreactive sites have a different retention time on C₁₈ resin in relation to oligonucleotides without the photoreactive site, the photoreactive probes may be isolated rapidly and conveniently in parallel purifications.

[2+2] Cycloaddition

According to the invention, [2+2] “cyclization,” “cyclodimerization,” or “cycloaddition” is a light-induced reaction between two photoreactive sites, at least one of which is electronically excited. Advantageously, [2+2] cycloaddition reactions can proceed with high efficiency. While it is chemical convention to write cycloaddition centers in brackets, such as “[2+2]” or “[4+2],” the brackets were omitted from the claims to prevent confusion with the patent convention of deleting bracketed material. Hence, in the claims “[2+2]” is written as “2+2.”

Most preferably, cycloaddition is of the [2+2] variety, wherein two carbon-carbon or a carbon-carbon and a carbon-heteroatom single bond are formed in a single step. The [2+2] cycloaddition involves addition of a 2π-component of a double bond to the 2π-component of a second double bond, as shown below.

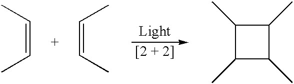

Alternatively, the reaction may proceed by way of a 2π-component of triple bonds. Under the rules of orbital symmetry, such [2+2] cycloadditions are thermally forbidden, but photochemically allowed. Such reactions typically proceed with a high degree of stereospecificity and regiospecificity.

Photochemical [2+2] cycloaddition of the probe to the hydrogel is obtained as follows. A first photoreactive site is chemically attached to the oligonucleotide with a phosphoramidite coupler to form a probe. A second photoreactive site is incorporated into the prepolymer or hydrogel following or as part of its polymerization, and prior to crosslinking. The combination is then irradiated with light at the appropriate wavelength to induce [2+2] cycloaddition, which results in the probe being covalently bound to the hydrogel.

Preferably, crosslinking occurs either prior to or simultaneously with probe attachment. Crosslinking of the prepolymer and probe attachment is preferably done with ultraviolet irradiation. Optionally, a photosensitiser may be added to the hydrogel or reactive prepolymer to increase the efficiency of the cycloaddition reaction. Preferred photosensitisers include water soluble quinones and xanthones, including anthroquinone, thioxanthone, sulfonic acid quinone, benzoin ethers, acetophenones, benzoyl oximes, acylphosphines, benzophenones, and TEMED (N,N,N′,N′-tetramethylethylendiamine). Anthroquinone-2-sulfonic acid is most preferred and is available from ALDRICH, Milwaukee, Wis.

Preferred [2+2] cycloadditions include those between two carbon-carbon double bonds to form cyclobutanes and those between alkenes and carbonyl groups to form oxetanes. Cycloadditions between 2 alkenes to form cyclobutanes can be carried out by photo-sensitization with mercury or directly with short wavelength light, as described in Yamazaki et al., J. Am. Chem. Soc., 91, 520 (1969). The reaction works particularly well with electron-deficient double bonds because electron-poor olefins are less likely to undergo undesirable side reactions. Cycloadditions between carbon-carbon and carbon-oxygen double bonds, such as α, β-unsaturated ketones, form oxetanes (Weeden, In Synthetic Organic Photochemistry, Chapter 2, W. M. Hoorspool (ed.) Plenum, New York, 1984) and enone addition to alkynes (Cargill et al., J. Org. Chem., 36, 1423 (1971)).

Photoreactive Sites

Photoreactive sites are defined as chemical bonds capable of undergoing [2+2] cycloaddition to form a ring structure when exposed to light of an appropriate wavelength. Photoreactive sites can yield homologous linking, where a probe photoreactive site cyclizes with a hydrogel photoreactive site having the same chemical structure, or for heterologous linking, where a probe photoreactive site cyclizes with a hydrogel photoreactive site having a different chemical structure. Preferred homologous linking occurs between dimethyl maleimide (DMI) photoreactive sites on the probe and hydrogel, while preferred heterologous linking occurs between cinnamide photoreactive sites on the probe and DMI photoreactive sites on the hydrogel. DNA is a preferred probe for either type of cyclization.

Preferable photoreactive sites may be provided by compounds including, dimethyl maleimide, maleimide, acrylate, acrylamide, vinyl, cinnamide groups from cinnamic acid, cinnamate, chalcones, coumarin, citraconimide, electron deficient alkenes such as cyano alkene, nitro alkene, sulfonyl alkene, carbonyl alkene, arylnitro alkene. Most preferred are cinnamide, and DMI. Other preferred photoreactive sites are as described in Guillet, Polymer Photophysics and Photochemistry, Ch. 12 (Cambridge University Press: Cambridge, London). Generally, any double bond that is not part of a highly conjugated system (e.g. benzene will not work) is preferred. Electron deficient double bonds, such as found in maleimide, are most preferred.

Additionally, molecules having a structure similar to dimethyl maleimide may be employed as photoreactive sites, including maleimide/N-hydroxysuccinimide (NHS) ester derivatives. Such preferred maleimide/NHS esters include 3-maleimidoproprionic acid hydroxysuccinimide ester; 3-maleimidobenzoic acid N-hydroxy succinimide; N-succinimidyl 4-malimidobutyrate; N-succinimidyl 6-maleimidocaproate; N-succinimidyl 8-maleimidocaprylate; N-succinimidyl 11-maleimidoundecaoate. These esters can be obtained from a variety of commercial vendors, such as ALDRICH (Milwaukee, Wis.).

Phosphoramidite Couplers

Phosphoramidite couplers may be used to attach multiple nucleosides to give oligonucleotides or to attach photoreactive sites to oligonucleotides. When used to attach photoreactive sites to oligonucleotides, probes are formed that may then be attached by a [2+2] cycloaddition to a polymer-support.

One procedure of synthesizing oligonucleotides using phosphoramidites involves attaching a nucleoside to a solid support, deprotecting the 5′-hydroxyl, and adding a phosphoramidite. The 5′-hydroxyl of the nucleoside attacks the phosphorous of the phosphoramidite, displacing the amine to form a phosphite triester. The phosphite is then oxidized to a phosphate triester, using 12 for example, and the 5′ protecting group removed from the nucleoside with an acid. A discussion of phosphoramidite monomers and their use as couplers in oligonucleotide synthesis is given in Bruice, Organic Chemistry, Ch. 25, pp. 1094-1096 (3^{rd} ed. 2001).

The generic structure of a phosphoramidite coupler is shown below. When used to synthesize nucleotides, one of the R groups is the nucleoside being added, routinely incorporating a protecting group. When used to incorporate a photoreactive site into an oligonucleotide, R′ or R″ can be the photoreactive site.

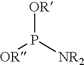

The phosphoramidite coupler may attach at the 5′ or 3′ position of the nucleoside. A 5′ attachment is depicted below.

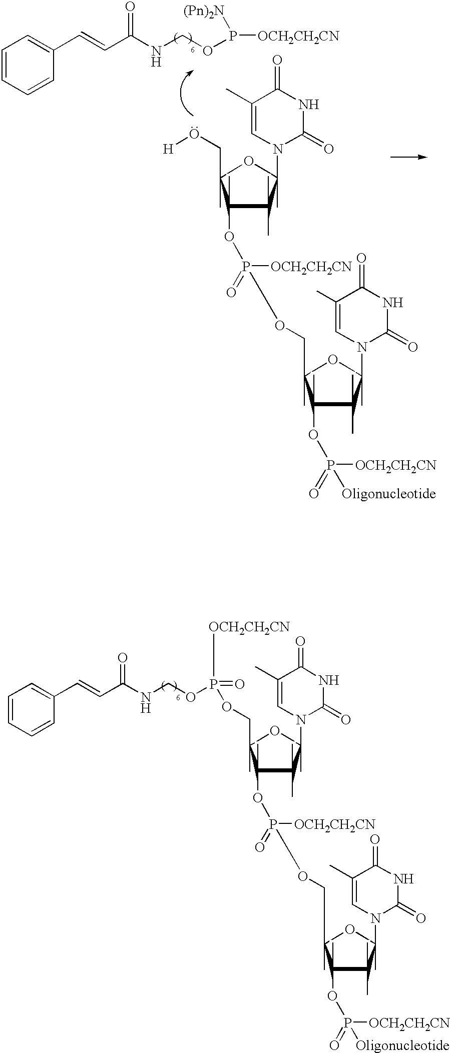

Preferably, a photoreactive probe is formed by providing a phosphoramidite coupler functionalized with a cinnamide photoreactive site, which is then attached to the oligonucleotide (5′ position for DNA) to form a probe ready for [2+2] cycloaddition. Additionally, other molecules having a functional group that will react with a phosphoramidite, including hydroxyl, thiol, and amine, can be attached to a photoreactive site and then coupled to a phosphoramidite coupler to form a useful photoreactive phosphoramidite.

Polymerization Resistance

Preferably, the first photoreactive site on the probe and the second photoreactive site on the polymer are resistant to chain-type polymerization. While some chain-type polymerization, as depicted below, is acceptable, the photoreactive phosphoramidites of the current invention reduce the occurrence of polymerization in relation to [2+2] cycloaddition.

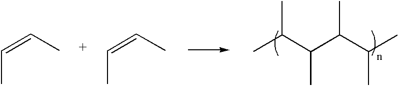

The disclosed photoreactive phosphoramidites reduce chain-type polymerization in relation to the desired [2+2] cycloaddition by suppressing the production of singlet oxygen and other radical species when irradiated with ultraviolet light. As an additional benefit, reduction of singlet oxygen generation reduces the formation of DNA-damaging hydroxy radicals, which is beneficial when oligonucleotides and other nucleic acid based probes are used.

Preferably, resistance to chain polymerization is imparted to the photoreactive site through the presence of one or more substituents attached to the double-bond carbons and/or because the photoreactive site double-bond is part of a ring structure. Electron-withdrawing substituents may be used to increase polymerization resistance. In this manner, the disclosed photoreactive phosphoramidites allow attachment of probes to solid supports, polymers, prepolymers, hydrogels, labels, and linkers through [2+2] cycloaddition, not polymerization reactions.

Photoreactive Phosphoramidites

Photoreactive phosphoramidite include phosphoramidite functionality and at least one photoreactive site capable of undergoing [2+2] cycloaddition when exposed to light of an appropriate wavelength. Preferred photoreactive phosphoramidites that are resistant to chain polymerization in which the photoreactive double bond is incorporated into a ring and di-methyl substituted are based on the following structure:

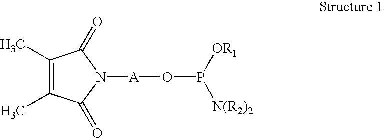

wherein A is an alkyl, cycloalkyl, cycloalkyl-alkyl, or heterocycloalkyl group. R₁ is any group that is compatible with oligonucleotide synthesis that may be removed after synthesis is complete. Preferably, R₁ is an alkyl or cycloalkyl group including at least one heteroatom. Most preferably, R₁ is —CH₂CH₂CN. The two R₂ groups may be the same or different and must also be capable of being bound to nitrogen and compatible with oligonucleotide synthesis. Preferably, the R₂ groups are alkyl, cycloalkyl, or alkyl groups that form a ring with the nitrogen, and may contain a second heteroatom (e.g. morpholino), most preferable are isopropyl groups.

The term “alkyl” refers to straight or branched saturated carbon chains substituted with hydrogen atoms. Examples of alkyl groups include methyl, ethyl, propyl, isopropyl, butyl, iso-, sec- and tert-butyl, pentyl, hexyl, heptyl, and 3-ethylbutyl. Similarly, “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl. “Cycloalkyl-alkyl” refers to a C₃-C₈ cycloalkyl group attached to a parent molecule through an alkyl group. Examples of cycloalkyl-alkyl groups include cyclopropylmethyl and cyclopentylethyl.

The term “heterocycloalkyl” refers to a cycloalkyl group containing at least one heteroatom selected from nitrogen, oxygen, and sulfur. The heterocycloalkyl ring may be attached to other rings and/or to a parent molecule through a carbon atom or a nitrogen atom. Preferred heterocycloalkyl groups have from 3 to 7 members and include piperidinyl, piperazinyl, morpholinyl, and pyrrolidinyl. “Heterocycloalkyl-alkyl” refers to a C₃-C₇ heterocycloalkyl attached to a parent molecule through an alkyl group.

Table 1 contains specific examples of A groups that result in useful di-β-methyl substituted photoreactive phosphoramidites when incorporated into Structure 1. The groups are incorporated at the bonds crossed by wavy lines.

TABLE 1

| Compound | A |
|---|---|
| 1 | 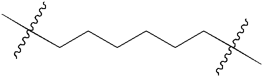 |
| 2 | 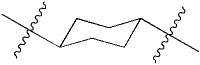 |
| 3 | 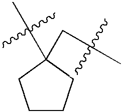 |
| 4 | 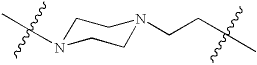 |

In addition to methyl substituted rings providing the photoreactive site double bond with resistance to chain polymerization as in Structure 1, chain-type polymerization resistance may also be imparted by incorporating an electron-withdrawing γ-substituent, if the photoreactive double-bond bridges the α-β-positions, as shown below.

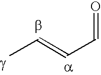

Structure 2, shown below, provides the molecular framework for preferred photoreactive phosphoramidites in which the photoreactive double-bond is incorporated into a ring structure and/or substituted with electron withdrawing substituents. D is an aryl, heteroaryl, cycloalkenyl, heterocycloalkenyl, cycloalkynyl, cycloalkylidenyl, or heterocycloalkylidenyl group.

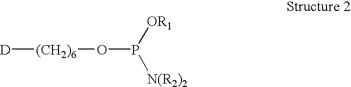

The term “aryl” refers to a hydrocarbon ring or ring system having at least one aromatic ring. The aromatic ring may optionally be fused or otherwise attached to other aromatic hydrocarbon rings or non-aromatic hydrocarbon rings. Preferred examples of aryl groups include phenyl and naphthyl.

“Heteroaryl” groups are aryl groups as defined above, but containing at least one heteroatom selected from nitrogen, oxygen, and sulfur. Examples of heteroaryl groups include, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, 4,5-dicyanoimidazole pyrazolyl, and benzopyrazolyl.

The term “alkenyl” refers to a straight or branched hydrocarbon containing at least one carbon-carbon double bond. Examples include vinyl, allyl, and 2-methyl-3-heptene. Similarly, “cycloalkenyl” refers to a C₃-C₈ cyclic alkenyl. Examples of cycloalkynyl groups include cyclopentene, cyclohexene and cycloheptene.

The term “heterocycloalkenyl” refers to a heterocyclic ring system containing one to three rings, wherein at least one ring is non-aromatic, the ring system contains at least one nitrogen, sulfur, or oxygen atom, and the ring system contains at least one non-aromatic carbon-carbon or carbon-nitrogen double bond. Examples of heterocycloalkenyl ring systems include, iminostilbene, 1,2-dihydroquinoline, 2-phenyl-3-methyl-3-pyrazolin-5-one, and pyrazole.

The term “cycloalkynyl” refers to a C₃-C₈ cyclic hydrocarbon containing at least one carbon-carbon triple bond. Examples of cycloalkynyl groups include cyclohexyne and cycloheptyne.

The term “cycloalkylidenyl” refers to a cycloalkyl di-radical wherein two carbon-hydrogen bonds are replaced independently with carbon-carbon, carbon-nitrogen, or carbon oxygen bonds. Cycloalkylidenyl groups include spiro-cyclic hydrocarbon ring systems. Examples of cycloalkylidenyl groups include, cis and trans cyclohexyl, cis and trans cyclopentyl, cis and trans cyclobutyl, and cis and trans cyclopropyl.

Similarly, the term “heterocycloalkylidenyl” refers to a cycloalkyl di-radical containing at least one heteroatom selected from nitrogen, oxygen, and sulfur, wherein two carbon-hydrogen bonds, two nitrogen-hydrogen bonds, or one carbon-hydrogen and one nitrogen-hydrogen bond has been replaced with two carbon-carbon bonds, two nitrogen-carbon bonds, or one carbon-carbon bond and one carbon-nitrogen bond. Examples of heterocycloalkylidenyl groups include, piperazinyl, homopiperazinyl, and methyl 3-amino-2-thiophenecarboxylate.

The terms “halogen” or “halo” indicate fluorine, chlorine, bromine, and iodine. The term “alkoxy” represents an alkyl group of indicated number of carbon atoms attached to the parent molecular moiety through an oxygen bridge. Examples of alkoxy groups include, for example, methoxy, ethoxy, propoxy and isopropoxy. The terms “hydroxyl,” and “hydroxy” refer to an —OH group and the term “amino” refers to a —NH₂ group.

Table 2 contains specific examples of D groups that result in useful phosphoramidites when incorporated into Structure 2. The groups are incorporated at the bonds crossed by wavy lines.

TABLE 2

| Compound | D |
|---|---|
| 5 | 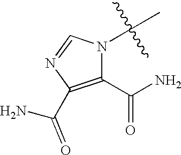 |
| 6 | 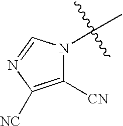 |
| 7 | 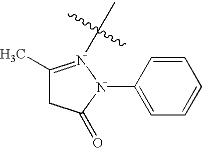 |
| 8 | 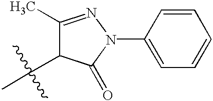 |
| 9 | 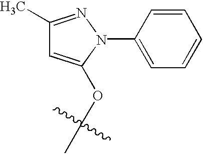 |
| 10 | 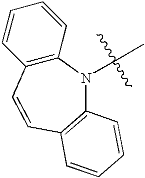 |
| 11 | 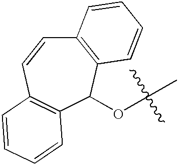 |
| 12 | 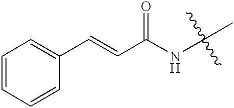 |
| 13 | 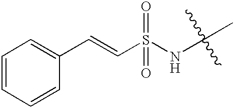 |
| 14 | 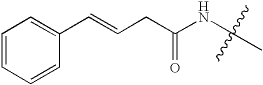 |

Linkers

The photoreactive phosphoramidites may optionally incorporate various linkers or linker regions. The linker region is a portion of the molecule which physically separates the photoreactive site, which undergoes [2+2] cycloaddition, from the remainder of the oligonucleotide. A linker region may also separate a photoreactive site from the polymer support. Although not wishing to be bound by any particular theory, it is thought that the linker region separates the oligonucleotide portion of the probe that is recognized by the target from the support, thus making the oligonucleotide more “available” for recognition by the target or enzyme.

Such linker regions are known and have been described in the art, and in some cases, may be commercially available, such as biotin (long arm) maleimide, available from GLEN RESEARCH, Sterling, Va., for example. Any linker region can be used, so long as the linker region does not negate the ability of the nucleic acid or oligonucleotide species to function as a probe. Preferred linker regions are organic chains of about 6 to 100 atoms long, such as (CH₂)₆ NH, (CH₂CH₂O)₅CH₂CH₂NH, etc. Additionally, linkers may be linked to each other, or to different types of linkers, to extend their chain length and may incorporate photoreactive sites capable of undergoing [2+2] cycloaddition with other photoreactive sites.

Photoreactive Phosphoramidite Synthesis

Many pathways exist to synthesize the photoreactive phosphoramidites of the current invention. Preferred methods are found in Reaction Schemes I through III.

Reaction Scheme I depicts the conversion of compounds (i) and (ii) to (iii) by combining (i), (ii), and a base in a solvent.

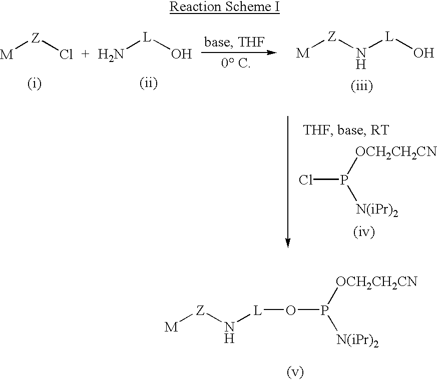

L is selected from C₁-C₈ alkyl, C₁-C₈ alkyl-C₃-C₈ cycloalkylidene-C₁-C₈ alkyl, C₁-C₈ alkyl-C₃-C₈ cycloalkylidenyl, C₃-C₈ cycloalkylidenyl, C₁-C₃ alkyl-C₃-C₈ heterocycloalkylidene-C₁-C₃ alkyl, C₁-C₃ alkyl-C₃-C₈ heterocycloalkylidenyl, and C₃-C₈ heterocycloalkylidenyl, wherein each of the above is optionally substituted with 1, 2 or 3 groups independently selected from the group consisting of C₁-C₄ alkyl, C₁-C₄ alkoxy, halogen, amino, mono- or di-C₁-C₄ alkylamino, trifluoromethyl, trifluoromethoxy, carboxamido, mono or di-C₁-C₄ alkyl-carboxamido, phenyl, C₁-C₄ alkoxycarbonyl, cyano, phenyl, and oxo (except that free hydroxy, amine, or thiol groups cannot be used). Most preferably, L is —C₆H₁₂—.

M is selected from C₁-C₈ alkenyl and C₁-C₈alkenyl-C₁-C₁₅ aryl. Most preferably, M is styrene or phenyl allyl.

Z is C═O or SO₂.

Specific examples of useful tertiary amine bases include triethylamine, diisopropylethylamine, lutidine, and pyridine. Specific examples of solvents include tetrahydrofuran (THF), dichloromethane (DCM), chloroform, diethyl ether, pyridine, 1,2-dimethoxyethane, and mixtures thereof. The reaction generally occurs at 0° C., although it may be proceed at temperatures as low as −40° C. or as high as reflux, the temperature depending on the specific solvent or solvents used in the reaction. The reaction time is generally about 30 minutes to about 36 hours.

The conversion of (iii) to (v) can be accomplished by treating (iii) with a phosphoramidite, and an additive in a solvent. Specific examples of phosphoramidites include chloro-(N,N-dimethyl-amino)methoxyphosphine, chloro-(2-cyanoethoxy)-N,N-diisopropyl-aminophosphine, and bis-(N,N-diisopropylamino)-2-cyanoethoxyphosphine. Specific examples of additives include 1H-tetrazole, N,N-diisopropylammonium tetrazolide, and dicyanoimidazole. Specific examples of solvents include acetonitrile, THF, DCM, chloroform, N,N-dimethylformamide, ethyl ether, 1,2-dimethoxyethane, and 1,4-dioxane. The reaction generally occurs at room temperature, although it may proceed at temperatures as low as −10° C. or as high as reflux. Typically, the reaction temperature depends on the specific solvent or solvents used for the reaction. The reaction time is generally about 30 minutes to about 16 hours.

Reaction Scheme II depicts the conversion of (vi) to (vii) by treating (vi) with a chloride source in a solvent. This reaction may be used to synthesize compound 9 from above.

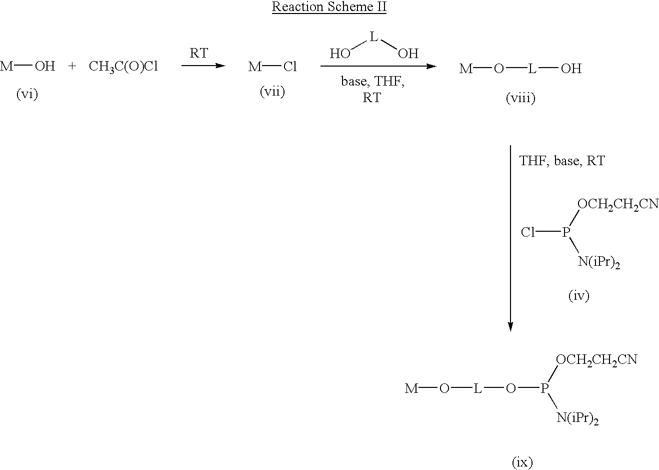

L is defied as in Scheme I above. M is aryl. Most preferably, (vi) is dibenzosuberenol.

Specific examples of chloride sources include acetyl chloride, SOCl₂, SO₂Cl₂, PCl₅, PCl₃, and HCl. Specific examples of solvents include acetyl chloride, THF, DCM, chloroform, diethyl ether, 1,4-dioxane, and mixtures thereof. The reaction generally occurs at room temperature, although it proceeds at temperatures as low as −78° C. or as high as reflux. Typically, the optimal temperature is dependant on the solvent or solvents used for the reaction. The reaction time is generally about 2 to about 36 hours.

The conversion of (vii) to (viii) can be accomplished by treating (vii) with a nucleophilic oxygen and a base in a solvent. Specific examples of nucleophilic oxygens include alcohols and carboxylate anions. More preferred are alcohols, including 1,6-dihydroxyhexane, 1,3-butanediol, 2-methyl-1,3-propanediol, and 1,4-cyclohexanediol. Specific examples of solvents include THF, 1,4-dioxane, methyl-t-butyl ether, diethylether and 1,2-dimethoxyethane. The reaction generally occurs at room temperature, although it proceeds at temperatures as low as −78° C. or as high as reflux. Typically, the optimal temperature is dependant on the solvent or solvents used for the reaction. Reaction time is generally about 2 to about 36 hours.

The conversion of (viii) to (ix) can be accomplished by treating (viii) with a phosphoramidite, and an additive in a solvent. Specific examples of phosphoramidites include chloro-(N,N-dimethyl-amino)methoxyphosphine, chloro-(2-cyanoethoxy)-N,N-diisopropyl-amino phosphine, and bis-(N,N-diisopropylamino)-2-cyanoethoxyphosphine. Specific examples of additives include 1H-tetrazole, N,N-diisopropylammonium tetrazolide, and dicyanoimidazole.

Specific examples of solvents include acetonitrile, THF, DCM, chloroform, N,N-dimethylformamide, ethyl ether, 1,2-dimethoxyethane, and 1,4-dioxane. The reaction generally occurs at room temperature, although it may be run at temps as low as −10° C. or as high as reflux, the temperature of which depends on the specific solvent or solvents used in the reaction. The reaction time is generally about 30 minutes to about 16 hours.

Reaction Scheme III depicts the conversion of (x) to (xi) by treating (x) with a base in a solvent and then adding an alkylating agent.

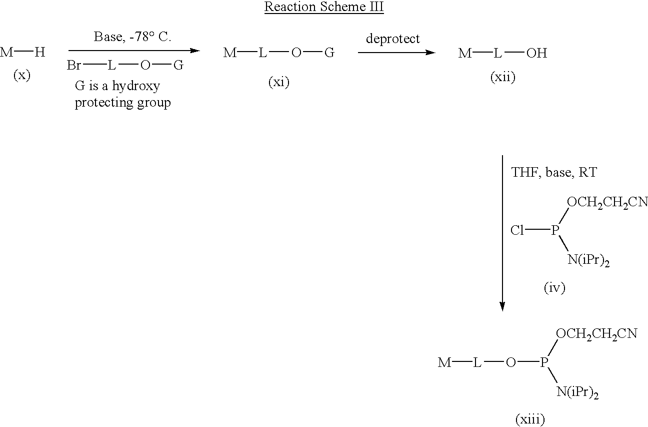

L is defined as in Scheme I above. M is selected from heteroaryl and heterocycloalkenyl. Optionally, the rings may be substituted with alkyl, oxo, aryl, and cyano groups. Preferably, (x) is di-cyano-imidazole, 2-phenyl-3-methyl-3-pyrazolin-5-one, or dimethyl maleimide. Di-cyano-imidazole and dimethyl maleimide are especially preferred as (x).

Specific examples of bases include lithium diisopropylamide, t-butyllithium, n-butyllithium, potassium hexamethyl disilylamide (KHMDS), lithium hexamethyldisilylamide (LiHMDS) and sodium hexamethyidisilylamide (NaHMDS). Specific examples of solvents include THF, 1,4-dioxane, diethylether, 1,2-dimethoxyethane, methyl-t-butyl ether (MTBE), hexamethylphosphoramide (HMPA), and mixtures thereof. Specific examples of alkylating agents include 6-bromohexyl-1-t-butyldimethylsilyl ether, N-Boc-4-iodo-2-methylaniline, and 3-bromopropoxy-1-t-butyldimethylsilane.

In general, the reaction is started at about −90° C. to −60° C., slowly warmed to room temperature, and optionally heated to reflux. The exact temperatures used depend on the solvent or solvents used in the reaction. Additionally, in some instances the reaction may be quenched at temperatures below room temperature. The reaction time is generally about 2 to about 48 hours.

The conversion of (xi) to (xii) can be accomplished by treating (xi) with an appropriate deprotecting agent in a solvent. Specific examples of deprotecting agents include, tetrabutylammonium fluoride, triethylammonium trihydrofluoride, trifluoroacetic acid, hydrogen chloride, hydrogen and palladium on carbon, hydrogen fluoride and aqueous sodium hydroxide. Specific examples of solvents include THF, DCM, chloroform, methanol, and diethyl ether. The reaction generally occurs at room temperature, but may proceed at temperatures as low as −10° C. or as high as reflux, the temperature of which depends on the specific solvent or solvents used in the reaction. The reaction time is generally about 30 minutes to about 48 hours.

The conversion of (xii) to (xiii) can be accomplished by treating (xii) with a phosphoramidite, and an additive in a solvent. Specific examples of phosphoramidites include chloro-(N,N-dimethyl-amino)methoxyphosphine, chloro-(2-cyanoethoxy)-N,N-diisopropyl-aminophosphine, and bis-(N,N-diisopropylamino)-2-cyanoethoxyphosphine.

Specific examples of additives include 1 H-tetrazole, N,N-diisopropylammonium tetrazolide, and dicyanoimidazole. Specific examples of solvents include acetonitrile, THF, DCM, chloroform, N,N-dimethylformamide, ethyl ether, 1,2-dimethoxyethane, and 1,4-dioxane. The reaction generally occurs at room temperature, although it may be run at temps as low as −10° C. or as high as reflux, the temperature of which depends on the specific solvent or solvents used in the reaction. The reaction time is generally about 30 minutes to about 16 hours.

The invention is illustrated further by the following non-limiting examples. Those of skill in the art will recognize that the starting materials may be varied and additional steps employed to produce compounds encompassed by the present inventions. In some cases, protection of certain reactive functionalities may be necessary to achieve some of the above transformations. In general, such need for protecting groups, as well as the conditions necessary to attach and remove such groups, will be apparent to those skilled in the art of chemistry.

### EXAMPLES

### Example 1

Preparation of Compounds 1-4

To form compound 1, 2,3-Dimethylmaleic anhydride and 6-amino-1-hexanol were heated in dry toluene until the water produced by the reaction had distilled. The toluene was evaporated and the residue partitioned between aqueous bicarbonate and ethyl acetate. The ethyl acetate was extracted with another portion of bicarbonate, then dried and evaporated to yield N-(6-hydroxyhexyl)-2,3-dimethylmaleimide. This conversion is depicted below.

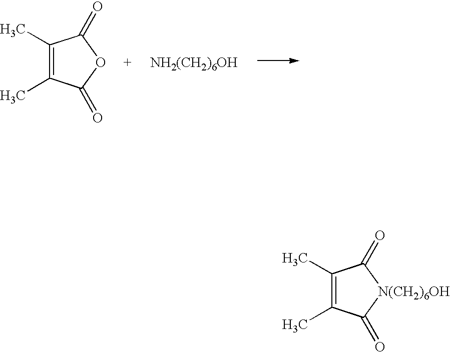

This alcohol was then reacted as described in Example 7 to give phosphoramidite Compound 1. Compounds 2-4 can be prepared in a similar fashion.

### Example 2

Preparation of Compounds 6, 7, and 9.

To form compound 6, 2,3-Dicyanoimidazole, 1-bromo-6-hexanol, and potassium carbonate were heated at 100° C. in DMF for three hours. The reaction mixture was cooled to room temperature and concentrated under reduced pressure. The concentrate was partitioned between water and ethyl acetate. The layers were separated and the aqueous layer extracted twice with ethyl acetate. The combined ethyl acetate layers were washed with 5% aqueous sodium bicarbonate, dried over sodium sulfate, filtered and concentrated. The residue was purified on silica gel using methanol/ethyl acetate as the eluant to afford the desired product. This conversion is depicted below.

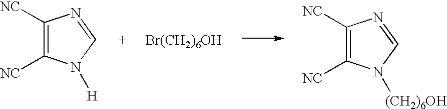

The product oil, 1-(6-hydroxyhexyl)-2,3-dicyanoimidazole, was converted to the phosphoramidite as described in Example 7 to give phosphoramidite Compound 6. Compounds 7-9 were prepared in a similar fashion.

### Example 3

Preparation of Compound 10 by Scheme III.

n-Butyl lithium in hexanes (0.05 mmol) was added to an orange, −78° C. solution of iminostilbene (0.05 mmol) in THF. Then, 6-Bromohexyl-1-t-butyldimethylsilyl ether (0.07 mmol) was added to the −78° C. reaction mixture, and the cooling bath was then removed. After warming to room temperature, the reaction mixture was refluxed for one hour. After cooling to room temperature, the reaction mixture was quenched with methanol slowly and extracted with ether. Chromatography on silica gel resulted in a yellow oil (Yield: 40%), compound (xi) from Scheme III.

Tetrabutylammonium fluoride (1M in hexanes, 1.0 mmol) was added to a 25° C. solution of the silyl ether (0.02 mmol) in THF. After stirring for 2 hours, the reaction mixture was quenched by addition of water followed by ether/5% sodium bicarbonate workup. The combined solvent layers were dried over Na₂SO₄, filtered and concentrated. The concentrate was purified by silica gel column chromatography to afford an oil, (Yield: 85%), compound xii from Scheme III. The compound was reacted as described in Example 7 to give phosphoramidite Compound 10.

### Example 4

Preparation of Compound 11.

Dibenzosuberenol (1.0 mmol) (vi) was dissolved in acetyl chloride and stirred at room temperature for ten hours to yield a clear, pale orange solution. The excess acetyl chloride was removed by evaporation under reduced pressure to yield the chloride (vii) as a pale yellow solid.

Diisopropylethylamine (1.5 mmol) was added to the resulting solid, followed by an excess of 1,6-dihydroxyhexane (3.0 mmol) in THF. After stirring at room temperature for one day, the pale yellow reaction mixture was quenched with 5% aqueous sodium bicarbonate solution and extracted several times with ether. The combined ether layers were dried over Na₂SO₄, filtered and concentrated to yield a yellow oil. Column chromatography on silica gel yielded a pale yellow oil (Yield: 43%). This oil was reacted in accordance with Example 7 to give phosphoramidite Compound 11.

### Example 5

Preparation of Compounds 12-14 by Scheme I.

A THF solution of 6-amino-1-hexanol (0.042 mmol) (ii) was added to a 0° C. solution including the desired acid chloride (0.02 mmol) (i) in dry THF. After stirring for 30 minutes, the ice bath was removed and the reaction mixture was stirred for an additional 2 hours. The reaction was quenched by the addition of saturated aqueous sodium bicarbonate and extracted several times with ethyl acetate. The combined ethyl acetate layers were dried over sodium sulfate, filtered and concentrated. The concentrate was crystallized from ethanol/water to yield a fluffy white solid (Yield: 45%). This compound was reacted in accordance with Example 7 to give the desired phosphoramidite compounds 12-14.

### Example 6

Preparation of Phosphoramidite Compounds (v), (ix), and (xii) in Schemes I, II, and III, Respectively.

Alcohols prepared via schemes I (alcohol iii), II (alcohol viii), and III (alcohol xii) were converted to the corresponding phosphoramidites using the general procedure below. The alcohol (0.01 mmol), 2-cyanoethyl diisopropylchlorophosphoramidite (0.015 mmol), and diisopropylethylamine (0.03 mmol) were stirred in THF at room temperature for two hours. The reaction mixture was quenched with 5% aqueous sodium bicarbonate and extracted several times with ethyl acetate containing a few drops of triethylamine. The combined ethyl acetate layers were dried over anhydrous sodium sulfate, filtered and concentrated. The concentrate was purified on silica gel to afford the desired phosphoramidite product (Yield: 83%).

### Example 7

General Oligonucleotide Synthesis.

Oligonucleotide synthesis was carried out on a Perceptive Biosystems oligonucleotide synthesizer (in the “DMT off” mode) using appropriate solid supports for the desired sequence of interest and conventional phosphoramidite chemistry. Coupling of the novel phosphoramidites with the oligonucleotides was carried out by syringe synthesis on the columns. The oligonucleotides were deprotected using concentrated ammonia solution at 55° C. and purified by HPLC.

What is claimed:

1. A photoreactive phosphoramidite, wherein said photoreactive phosphoramidite incorporates a first photoreactive site that undergoes 2+2 cycloaddition with a second photoreactive site when irradiated with light of an appropriate wavelength.

2. A photoreactive phosphoramidite having the structure 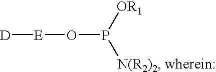
E is selected from the group consisting of C₁-C₈ alkyl, C₁-C₈ alkyl-C₃-C₈ cycloalkylidene-C₁-C₈ alkyl, C₁-C₃ alkyl-C₃-C₈ cycloalkylidenyl, C₃-C₈ cycloalkylidenyl, C₁-C₃ alkyl-C₃-C₈ heterocycloalkylidene-C₁-C₃ alkyl, C₁-C₃ alkyl-C₃-C₈ heterocycloalkylidenyl, and C₃-C₈ heterocycloalkylidenyl, wherein each of the above is optionally substituted with 1, 2 or 3 groups independently selected from the group consisting of C₁-C₄ alkyl, C₁-C₄ alkoxy, halogen, hydroxy, trifluoromethyl, trifluoromethoxy, amino, mono- or di-C₁-C₄ alkylamino, carboxamido, and mono- or di-C₁-C₄ alkyl-carboxamido;
D is selected from the group consisting of —R₃-C₅-C₁₀ cycloalkynyl, C₅-C₁₀ cycloalkynyl, C₆-C₁₈ aryl, —R₃-C₆-C₁₈ aryl, heteroaryl, C₃-C₁₄ heterocycloalkenyl, —R₃-C₃-C₁₄ heterocycloalkenyl, —R₃C(O) alkenyl, —R₃ alkenyl, wherein each of the above is optionally substituted with 1, 2, or 3 substituents independently selected from the group consisting of C₁-C₄ alkyl, C₁-C₄ alkoxy, halogen, amino, mono- or di- C₁-C₄ alkylamino, trifluoromethyl, trifluoromethoxy, carboxamido, mono- or di- C₁-C₄ alkyl-carboxamido, phenyl, C₁-C₄ alkoxycarbonyl, cyano, and oxo, wherein R₃ is O, NH, NHSO₂ and SO₂;
R₁ is an alkyl or a cycloalkyl group comprising a heteroatom; and
R₂ are the same or different and are alkyl groups that form a ring with the nitrogen or are independently selected alkyl, cycloalkyl, or heterocycloalkyl groups.

3. The photoreactive phosphoramidite of claim 2, wherein E is alkyl

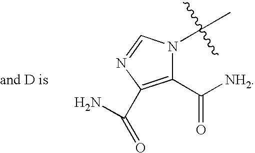

4. The photoreactive phosphoramidite of claim 2, wherein E is alkyl

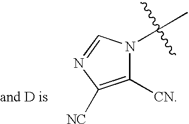

5. The photoreactive phosphoramidite of claim 2, wherein E is alkyl

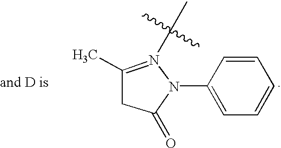

6. The photoreactive phosphoramidite of claim 2, wherein E is alkyl

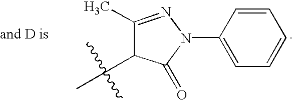

7. The photoreactive phosphoramidite of claim 2, wherein E is alkyl

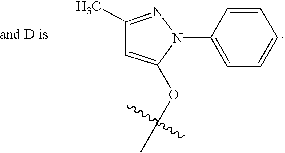

8. The photoreactive phosphoramidite of claim 2, wherein E is alkyl

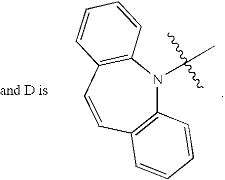

9. The photoreactive phosphoramidite of claim 2, wherein E is alkyl

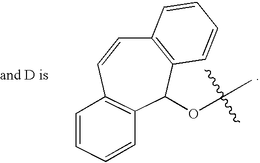

10. The photoreactive phosphoramidite of claim 2, wherein E is alkyl

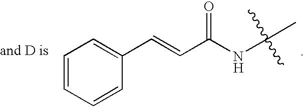

11. The photoreactive phosphoramidite of claim 2, wherein E is alkyl

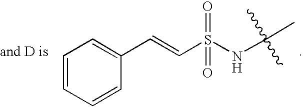

12. The photoreactive phosphoramidite of claim 2, wherein E is alkyl

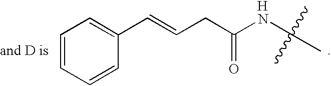

13. The photoreactive phosphoramidite of claim 2, wherein R₁ is —CH₂CH₂CN and R₂ is isopropyl.

14. A photoreactive phosphoramidite having the structure 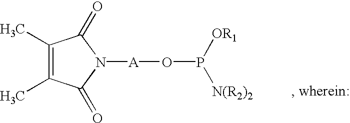
A is selected from the group consisting of C₄-C₈ alkyl, C₃-C₈ cycloalkyl, C₃-C₈ cycloalkyl-C₁-C₃ alkyl, C₃-C₈ heterocycloalkyl, C₃-C₈ heterocycloalkyl-C₁-C₃ alkyl, wherein A is optionally substituted with one or more groups independently selected from halogen, C₁-C₄ alkyl, C₁-C₄ alkoxy, amino, hydroxy, mono- or di- C₁-C₄ amino;
R₁ is an alkyl or a cycloalkyl group comprising a heteroatom; and
R₂ are the same or different and are alkyl groups that form a ring with the nitrogen or are independently selected alkyl, cycloalkyl, or heterocycloalkyl groups.

15. The photoreactive phosphoramidite of claim 14, wherein A is hexyl.

16. The photoreactive phosphoramidite of claim 14, wherein A is cyclohexyl.

17. The photoreactive phosphoramidite of claim 14, wherein A is

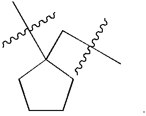

18. The photoreactive phosphoramidite of claim 14, wherein A is

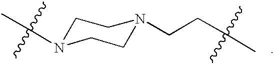

19. The photoreactive phosphoramidite of claim 14, wherein R₁ is −CH₂CH₂CN and R₂ is isopropyl.

20. A photoreactive phosphoramidite having the structure 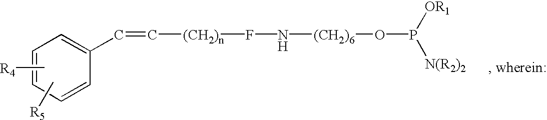
n is 0, 1, 2, or 3;
F is either —C(O)— or —SO₂—;
R₄ and R₅ are the same or different and are independently selected from the group consisting of hydrogen, C₁-C₄ alkyl, C₁-C₄ alkoxy, hydroxy, trifluoromethyl, nitro, halo, and phenyl;
R₁ is an alkyl or a cycloalkyl group comprising a heteroatom; and
R₂ are the same or different and are alkyl groups that form a ring with the nitrogen or are independently selected alkyl, cycloalkyl, or heterocycloalkyl groups.

21. The photoreactive phosphoramidite of claim 20, wherein R₁ is —CH₂CH₂CN and R₂ is isopropyl.

22. A probe for use in a microarray analysis comprising a photoreactive site and an oligonucleotide, wherein said photoreactive site is incorporated into said oligonucleotide by a photoreactive phosphoramidite.

23. The probe of claim 22, wherein the microarray analysis is an expression or a SNP analysis.

24. The probe of claim 22, wherein said photoreactive phosphoramidite is the photoreactive phosphoramidite of claim 14.

25. The probe of claim 22, wherein said photoreactive phosphoramidite is the photoreactive phosphoramidite of claim 2.

26. The probe of claim 22, wherein said photoreactive phosphoramidite is the photoreactive phosphoramidite of claim 20.

27. A composition comprising an oligonucleotide covalently attached to a hydrogel, wherein said attachment is by a 2+2 cycloaddition between a first photoreactive site incorporated into said oligonucleotide by a photoreactive phosphoramidite and a second photoreactive site present on said hydrogel.

28. The composition of claim 27, wherein said photoreactive phosphoramidite is the photoreactive phosphoramidite of claim 14.

29. The composition of claim 27, wherein said photoreactive phosphoramidite is the photoreactive phosphoramidite of claim 2.

30. The composition of claim 27, wherein said photoreactive phosphoramidite is the photoreactive phosphoramidite of claim 20.

31. A method of making a photoreactive oligonucleotide probe comprising:
(a) a stepwise addition of nucleoside phosphoramidites to a synthesis support to form an oligonucleotide;
(b) chemically coupling a photoreactive phosphoramidite with the oligonucleotide to form the photoreactive oligonucleotide probe, wherein said photoreactive oligonucleotide probe incorporates a first photoreactive site capable of undergoing 2+2 cycloaddition;
(c) removing the photoreactive oligonucleotide probe from the synthesis support; and
(d) optionally deprotecting and purifying said photoreactive oligonucleotide probe.

32. The method of claim 31, wherein said photoreactive phosphoramidite is the photoreactive phosphoramidite of claim 14.

33. The method of claim 31, wherein said photoreactive phosphoramidite is the photoreactive phosphoramidite of claim 2.

34. The method of claim 31, wherein said photoreactive phosphoramidite is the photoreactive phosphoramidite of claim 20.

35. A method of attaching the photoreactive oligonucleotide probe of claim (c) to a hydrogel comprising:
(a) providing the photoreactive oligonucleotide probe;
(b) providing a hydrogel with a second photoreactive site;
(c) covalently bonding said photoreactive oligonucleotide probe to said hydrogel by combining said photoreactive oligonucleotide probe with said hydrogel and irradiating with ultraviolet light, wherein a 2+2 cycloaddition occurs between the first and second photoreactive sites.

36. The method of claim 35, wherein said photoreactive phosphoramidite is the photoreactive phosphoramidite of claim 14.

37. The method of claim 35, wherein said photoreactive phosphoramidite is the photoreactive phosphoramidite of claim 2.

38. The method of claim 35, wherein said photoreactive phosphoramidite is the photoreactive phosphoramidite of claim 20.

39. In a method for synthesizing an oligonucleotide or nucleic acid probe for attachment to a hydrogel, the improvement comprising incorporating a photoreactive site into a phosphoramidite coupler to form a photoreactive phosphoramidite and reacting said photoreactive phosphoramidite with said oligonucleotide or nucleic acid.

40. The method of claim 39, wherein said photoreactive phosphoramidite is the photoreactive phosphoramidite of claim 14.

41. The method of claim 39, wherein said photoreactive phosphoramidite is the photoreactive phosphoramidite of claim 2.

42. The method of claim 39, wherein said photoreactive phosphoramidite is the photoreactive phosphoramidite of claim 20.
